(19) United States
(12) Patent Application Publication
(10) Pub. No.: US 2014/0121647 A1
(43) Pub. Date: May 1, 2014

(54) SYSTEM FOR DISPENSING MULTIPLE FLUIDS AND A METHOD FOR USING THE SAME

(71) Applicant: NORDSON CORPORATION, Westlake, OH (US)

(72) Inventor: Thomas A. Kirk, Hastings, MN (US)

(73) Assignee: NORDSON CORPORATION, Westlake, OH (US)

(21) Appl. No.: 14/134,045

(22) Filed: Dec. 19, 2013

Related U.S. Application Data
(63) Continuation-in-part of application No. 13/777,507, filed on Feb. 26, 2013.
(60) Provisional application No. 61/719,991, filed on Oct. 30, 2012.

(51) Int. Cl. A61M 5/19

(52) U.S. Cl. CPC A61M 5/19
USPC 604/518; 604/191; 604/82

(57) ABSTRACT

A system for storing and dispensing first and second fluids. A syringe, including a first fluid chamber, a second fluid chamber, a first outlet communicating with the first fluid chamber and extending along a first axis; a second outlet communicating with the second fluid chamber and extending along a second axis, the second axis spaced from the first axis by a first distance. A container having first and second compartments separated by at least one wall and configured to store the first and second fluids, respectively, the first compartment having a first port positioned to be fluidicly connected with the first outlet and the second compartment having a second port positioned to be fluidicly connected with the second outlet.

### CROSS-REFERENCE TO RELATED APPLICATIONS

This application is a continuation-in-part of U.S. patent application Ser. No. 13/777,507, filed on Feb. 26, 2013 (pending), which claims the priority of U.S. Provisional Patent Application Ser. No. 61/719,991, filed on Oct. 30, 2012 (expired), the disclosures of which are incorporated by reference herein.

### TECHNICAL FIELD

The disclosure relates generally to dispensing multiple fluids with a system including a syringe.

### BACKGROUND OF THE INVENTION

Generally, it is well known to dispense multiple fluids from a syringe or other device. There are challenges and problems associated with filling multiple chambers of syringes with their respective fluids. For example, the fluids may be reactive such that interaction between the fluids before being dispensed would render the fluids unusable or cause the fluids to coagulate or cure such that dispensing the fluids from the syringe is impossible. Therefore, it is undesirable in many applications, such as with certain medical fluids and multi-part adhesives, to simply pour multiple fluids from a container into respective fluid chambers of a syringe, due to the risk of spillage, splashing, and other occurrences that would lead to undesired interaction between the fluids. Moreover, there may be situations where simply pouring multiple fluids into their respective fluid chambers is impossible. For example, the design of the syringe may be such that one or more of the chambers may not be accessible from the rear open end of the syringe. In that instance, the fluids may be drawn into respective chambers from separate containers, but the issues mentioned above still exist. Moreover, due to the dimensioning of the syringe or of the containers containing the fluids, it is typically not possible to simultaneously draw multiple fluids into fluid chambers of the syringe without the use of additional fluidic connectors, such as tubing. The use of extra equipment, however, introduces fluid mechanics issues and increases the risk of undesired and/or premature interaction of the fluids. There is therefore a need in the art to address these and other challenges in the art.

### SUMMARY

In an illustrative embodiment, the invention provides a system for dispensing first and second fluids including a syringe and a container. The syringe includes a first fluid chamber and a second fluid chamber. A first outlet communicates with the first fluid chamber and a second outlet communicates with the second fluid chamber. A transmission structure is provided and draws the first and second fluids into the first and second outlets and further into the first and second fluid chambers. The transmission structure is also used to dispense the first and second fluids from the first and second outlets. The container includes first and second compartments separated by at least one wall and is configured to store the first and second fluids. The first compartment includes a first port positioned to be fluidicly connected with the first outlet and the second compartment includes a second port positioned to be fluidicly connected with the second outlet.

The first outlet may extend along a first axis and the second outlet may extend along a second axis. The second axis is spaced from the first axis by a first distance, and the first and second ports are spaced apart by the first distance. A cannula assembly may be coupled with and fluidicly communicate with the first and second outlets. The cannula assembly includes a tip at a distal portion adapted to dispense the first and second fluids from the cannula assembly.

The first and second compartments may be configured to be releasably attached such that the container may be selectively assembled and disassembled. For example, the first and second containers may be coupled together with a snap fit. The volume of the first compartment may be greater than or equal to the volume of the first fluid chamber, and the volume of the second compartment may be greater than or equal to the volume of the second fluid chamber. At least one splash guard may be positioned between the first and second ports to prevent unintended mixing of the first and second fluids. At least one of the first or second ports may include a first open end communicating with an area outside of its respective compartment, a second open end communicating with its respective compartment, and a lumen therebetween. The second open end of at least one of the first or second ports may be substantially aligned with or adjacent to a deepest point of its respective first or second compartment. The lumen may extend along an axis that intersects a deepest point of its respective first or second compartment.

In another aspect, the invention provides a method of dispensing first and second fluids using a system including a syringe and a container. The syringe includes a first fluid chamber and a second fluid chamber, and the container includes first and second compartments respectively holding the first and second fluids. The method includes coupling the first chamber in fluid communication with the first compartment, and coupling the second chamber in fluid communication with the second compartment. The first fluid is drawn from the first compartment into the first chamber while the second fluid is drawn from the second compartment into the second chamber. The first and second chambers are decoupled from the first and second compartments, and the first and second fluids are simultaneously dispensed from the first and second chambers.

The method can include additional steps, such as releasably attaching the first and second compartments together prior to simultaneously drawing the first and second fluids. Releasably attaching the first and second compartments may include snap fitting the first and second compartments together. Drawing the first and second fluids from the first and second compartments can further include positioning a first open end of a first lumen at a deepest point of the first compartment, and positioning a second open end of a second lumen at a deepest point of the second compartment. Then, the first and second fluids are simultaneously drawn through the respective first and second lumens from the deepest points of the first and second compartments. Coupling the first and second chambers in fluid communication with the first and second compartments can further include coupling a first outlet of the syringe to a first port of the container extending along a first axis, and coupling a second outlet of the syringe to a second port of the container extending along a second axis.

These and other features of the various embodiments of this invention will become more readily apparent to those of ordinary skill upon review of the following detailed description of the illustrated embodiments taken in conjunction with the accompanying drawings.

### BRIEF DESCRIPTION OF THE DRAWINGS

FIG. 1 is a perspective view of a system for storing and dispensing multiple fluids.

FIG. 2A is a cross-sectional side view of a system for storing and dispensing multiple fluids in a filled position.

FIG. 2B is a cross-sectional side view of a system for storing and dispensing multiple fluids as the fluids are being drawn out of the system.

FIG. 2C is a cross-sectional side view of a system for storing and dispensing multiple fluids in a generally empty position.

FIG. 3A is a detailed cross-sectional side view of a system for storing and dispensing multiple fluids showing a vent.

FIG. 3B is a detailed cross-sectional side view of a system for storing and dispensing multiple fluids showing a vent fluidicly communicating with a cavity.

FIG. 4 is a perspective view of alternative embodiment of the system of FIG. 1 being pneumatically driven.

FIG. 5A is a cross-sectional side view of the system of FIG. 4 in a filled position.

FIG. 5B is a cross-sectional side view of the system of FIG. 4 in a generally empty position.

FIG. 6A is a cross-sectional side view of an alternative embodiment of a system for storing and dispensing multiple fluids in a filled position.

FIG. 6B is a cross-sectional side view of an alternative embodiment of a system for storing and dispensing multiple fluids in a generally empty position.

FIG. 7 is a container for holding first and second fluids, shown disassembled.

FIG. 8 is a cross-sectional view of one compartment of the container of FIG. 7.

FIG. 9 is a top view of the container of FIG. 7.

FIG. 10A is a cross-sectional view of a system for dispensing first and second fluids, including a syringe and the container of FIG. 7, with the syringe shown in an empty position.

FIG. 10B is a cross-sectional view of a system for dispensing first and second fluids, including a syringe and the container of FIG. 7, with the syringe shown in an intermediate position.

FIG. 10C is a cross-sectional view of a system for dispensing first and second fluids, including a syringe and the container of FIG. 7, with the syringe shown in a filled position.

### DETAILED DESCRIPTION

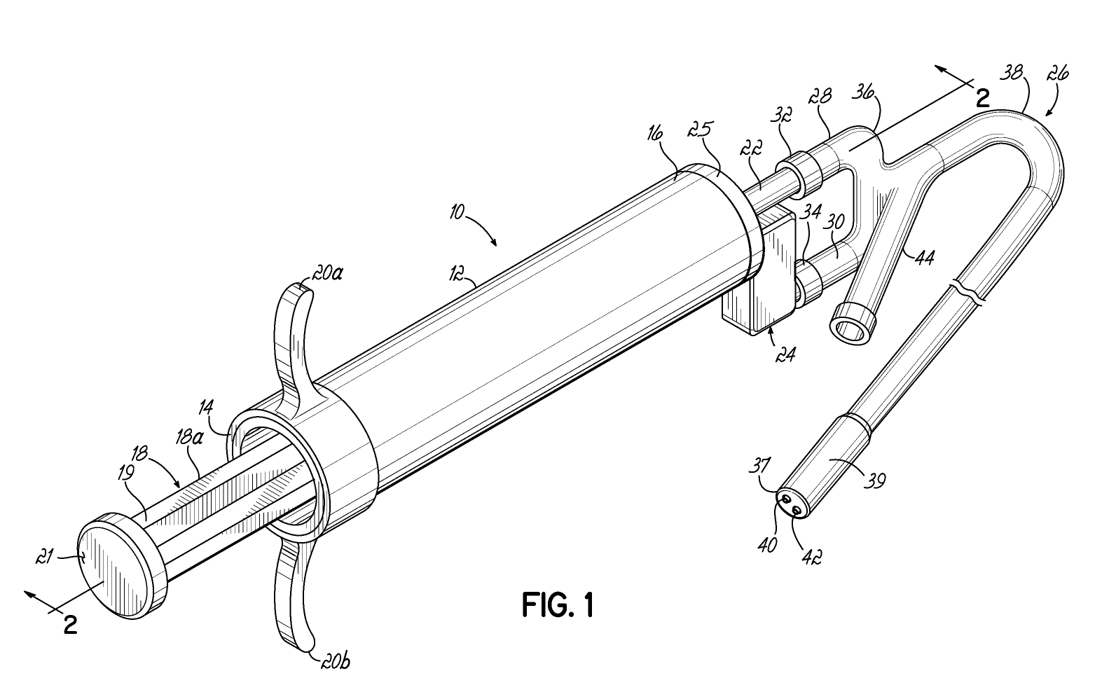

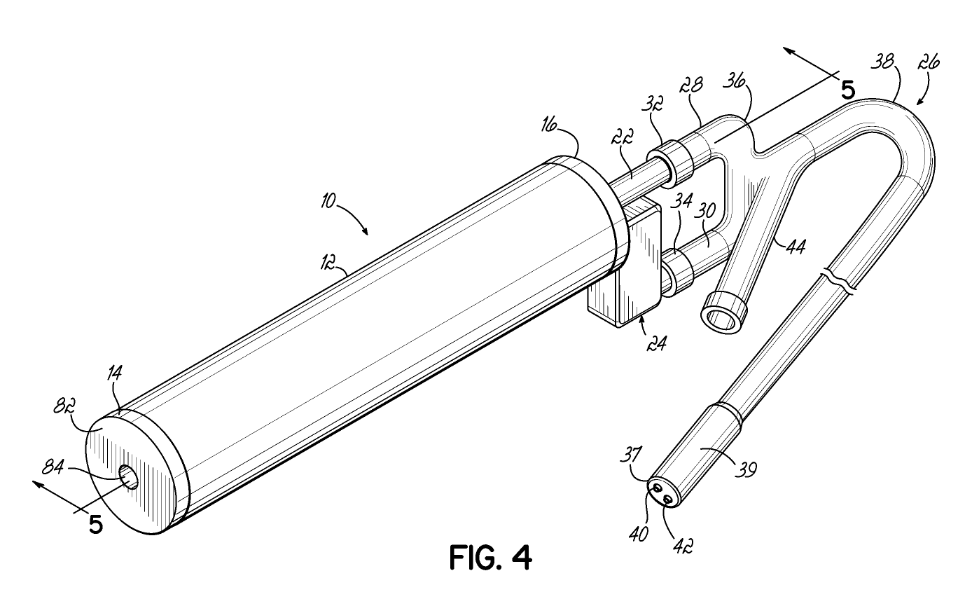

FIG. 1 shows a preferred embodiment of the system. The system comprises a syringe 10 including a barrel 12, the barrel 12 having a proximal end 14, a distal end 16 and a generally cylindrical shape. The proximal end 14 is open to accept a plunger or transmission structure 18. The transmission structure 18 preferably has a shaft 19 and a depressing surface 21 against which a user presses a thumb or other finger to advance the transmission structure 18 distally. In an alternative embodiment as seen in FIG. 4, the syringe 10 includes a pneumatic adaptor 82 adapted to advance the internal components of the barrel 12 instead of a plunger 18a. The barrel 12 preferably has finger grips 20a, 20b extending radially therefrom in order to assist the user in depressing the plunger 18a. In a preferred embodiment, the finger grips 20a, 20b comprise two members extending radially from the barrel 12. However, the finger grips 20a, 20b may alternatively comprise a radially extending flange or another configuration that assists the user in depressing the plunger 18a.

The distal end 16 of the barrel 12 has first and second outlets 22, 24 extending therefrom. The first and second outlets 22, 24 are fluidicly connected to a cannula assembly 26. Specifically, the first outlet 22 is connected to a first tube 28 and the second outlet 24 is connected to a second tube 30 of the cannula assembly 26. In a preferred embodiment, a portion of the second outlet 24 extends radially from the first outlet 22 in order to provide ample space for the connection with the first and second tubes 28, 30 of the cannula assembly 26. The first and second tubes 28, 30 each include adaptors 32, 34 configured to accept the first and second outlets 22, 24 and provide a seal between the first and second outlets 22, 24 and the first and second tubes 28, 30, respectively. There are first and second lumens (not shown) within the first and second tubes 28, 30, respectively. At the proximal end 36 of the cannula assembly 26, the first and second tubes 28, 30 bifurcate in order to facilitate a connection with the first and second outlets 22, 24. More distally along the cannula assembly 26, the first and second tubes 28, 30 converge into a main cannula 38. Within the main cannula 38 the first and second fluids that are discharged from the first and second outlets 22, 24 remain separated in first and second lumens (not shown) within the main cannula 38. The first and second lumens are fluidicly connected with the first and second tubes 28, 30. When the transmission structure 18 is depressed distally, the first and second fluids are discharged from the first and second outlets 22, 24, into and through the first and second tubes 28, 30 and into the first and second lumens. The first and second fluids may be, for example, biomedical materials used in a medical procedure. The first and second fluids travel within the first and second lumens within the main cannula 38 until reaching the tip 39 at the distal end 37 of the cannula assembly 26, whereupon the first and second fluids are discharged from first and second apertures 40, 42 of the tip 39. Extending radially from the main cannula 38 is an additional tube 44 adapted to communicate with a gas source. In a preferred embodiment, the first aperture 40 is situated at least partially transverse to the second aperture 42, whereby the first and second fluids interact after being dispersed from the first and second apertures 40, 42. Preferably, an interaction between the first and second fluids creates a third material comprising the first and second fluids.

Due to the configuration of tube 44 and cannula assembly 26, the system is operative to dispense the first and second fluids from the first and second apertures 40, 42 by the aid of a pressurized gas. In a preferred embodiment, the first and second fluids are mixed in flight after being dispensed with the aid of a pressurized gas from the first and second apertures 40, 42. The mixture of the first and second fluids is deposited onto a biological substrate such as a portion of the human body.

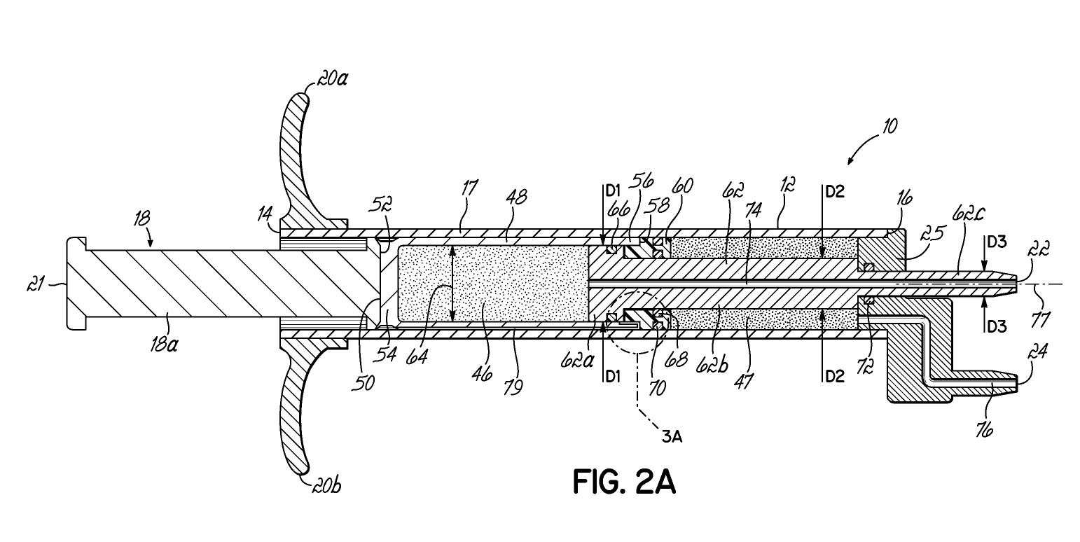

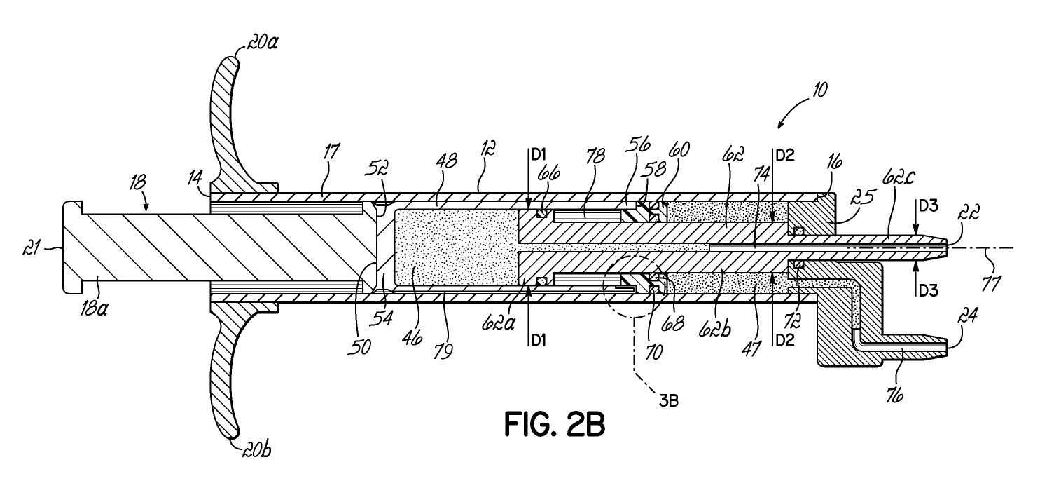

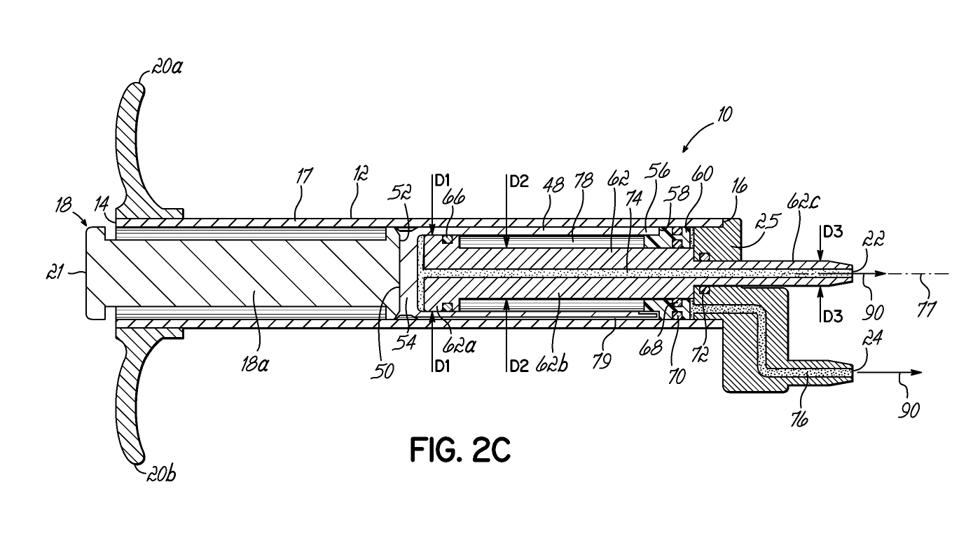

FIG. 2A shows the syringe 10 in a filled position. In a preferred embodiment, first and second fluid chambers 46, 47 are at least partially filled with first and second fluids. Preferably, transmission structure 18 is disposed within the barrel 12 at the proximal end 14, and the tip adaptor 25 is disposed within the barrel 12 at the distal end 16 prior to being packaged and sent to a clinical environment for use. The tip adaptor 25 closes and creates a seal at the distal end 16. Tip adaptor may be fixedly coupled to the distal end 16 of barrel 12 by various methods of manufacture known to persons skilled in the art. Preferably, the tip adaptor 25 is bonded to barrel 12 using an adhesive. Preferably, first and second fluid chambers 46, 47 are pre-filled with first and second fluids before being packaged and sent to a clinical environment for use. The system, however, may be packaged as unassembled and can be assembled and filled with first and second fluids on-site.

First piston 48 has a contact surface 50 at a first end 54 for accepting the transmission structure 18. The contact surface 50 and the contacting portion 52 of the transmission structure 18 contacting the contact surface 50 may have complimentary shapes for efficiency and ergonomics purposes. The first piston 48 may have a hollow, generally cylindrically shaped body extending distally from the contact surface 50 for forming part of the first fluid chamber 46. The second end 56 of the first piston 48 is open and includes a contact surface 58 that engages with the second piston 60. More specifically, the second end 56 is bonded to the second piston 60 generally at the location of the second end 56, such as by welding.

The delivery tube 62 may be fixedly, concentrically disposed relative to and within the barrel 12. In a preferred embodiment, the delivery tube 62 is coupled with the tip adaptor 25, thereby preventing the movement of delivery tube 62. The delivery tube may be fixed within the tip adaptor according to methods known to persons skilled in the art, such as sonic welding or bonding using adhesives. However, the manner of coupling is not limited to these methods and may be done so in other manners known to persons skilled in the art. The delivery tube 62 may have first, second and third sections 62a, 62b, 62c having first, second and third diameters D1, D2 and D3, respectively. The third section 62c may have a tapered portion at or near the first outlet 22 to enable the third section 62c to engage with a downstream device such as a luer taper, tubing or a cannula assembly 26 (see FIG. 1). Preferably, the first diameter D1 is larger than the second D2 and third D3, and the second diameter D2 is larger than the third diameter D3. In alternative embodiments, the first, second, and third diameters D1, D2 and D3 may vary, especially where the volume ratio of the first and second fluids are changed (as discussed below). The delivery tube 62 includes a first channel 74 disposed along the length and concentrically relative to first, second and third sections 62a, 62b, 62c.

The first diameter D1 and the inner diameter 64 of first piston 48 may be substantially the same, thereby creating a seal between the first piston 48 and delivery tube 62. In order to create an effective seal, a first O-ring 66 may be disposed on the delivery tube 62. The seal created by the first O-ring 66 may prevent the first fluid from the first chamber 46 to leak into other parts of the barrel 12, such as the cavity 78 that forms as the first and second pistons 48, 60 move distally. See FIGS. 2B, 2C. In an unengaged position, as seen in FIG. 2A, the first end 54 of the first piston 48 is proximal to the delivery tube 62, thereby defining a first fluid chamber 46 between the second end 56 and the first section 62a of the delivery tube 62. The second piston 60 is at least partially disposed between the wall 17 of the barrel 12 and the second section 62b of the delivery tube 62. The second piston 60 has second and third O-rings 68, 70 disposed thereon. The second O-ring 68 provides a seal between the second section 62b of the delivery tube 62 and the second piston 60. The third O-ring 70 provides a seal between the second piston 60 and the wall 17 of the barrel 12. The seals provided by the second and third O-rings 68, 70 prevent the first fluid from escaping the second chamber 47 and entering other parts of the barrel 12, such as the cavity 78.

In the unengaged or filled state as seen in FIG. 2A, the transmission structure 18 may be pushed to cause distal movement of first piston 48 and thereby distal movement of the second piston 60 as well. The third section 62c of the delivery tube 62 is concentrically disposed relative to and passes through an aperture in the tip adaptor 25. To provide for an effective seal, there is a fourth O-ring 72 disposed on the tip adaptor 25 concentrically relative to the third section 62c of the delivery tube 62. The fourth O-ring 72 contacts the third section 62c of the delivery tube 62 and creates a seal, thereby preventing the second fluid from leaving the second fluid chamber 47 in an unwanted manner, such as through the aperture in the tip adaptor 25. Tip adaptor 25, second piston 60, wall 17 and delivery tube 62 define the boundaries for the second fluid chamber 47. The second fluid chamber 47 fluidicly connects with the second outlet 24 by way of the second channel 76. In one embodiment, at least one of the first or second outlets 22, 24 is disposed radially outward relative to a central axis 77 of the barrel. For example, in the embodiment shown in FIGS. 2A-B, the second outlet 24 is disposed radially outward from the central axis 77 of the barrel while the first outlet 22 is disposed along the axis 77.

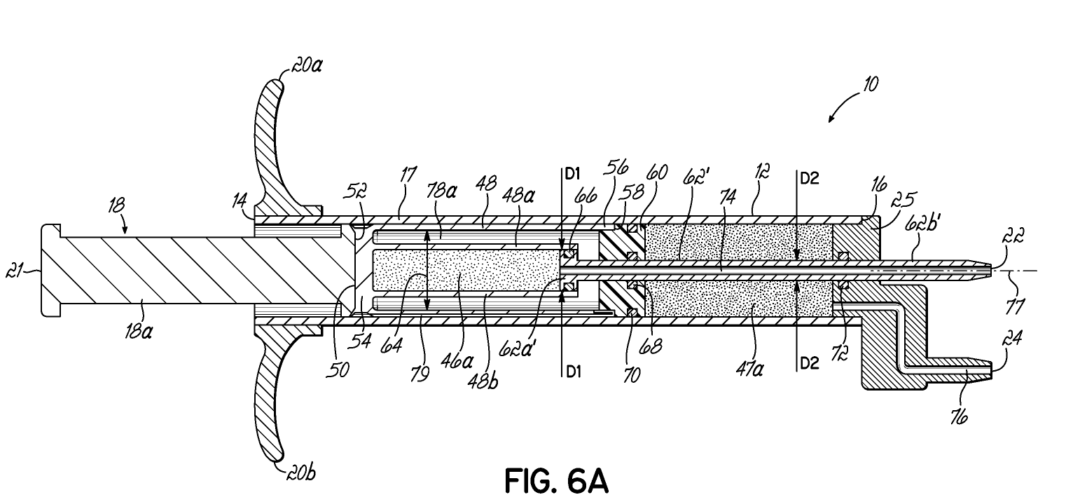

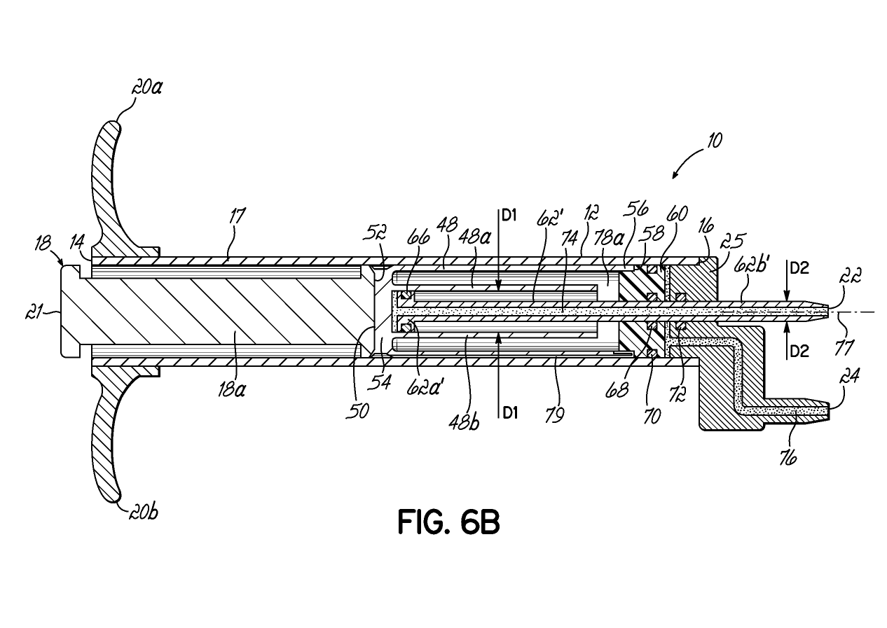

FIG. 2B shows the system in a position between the filled position and an empty position. Upon the distal movement of the transmission structure 18, the first end 54 of the first piston 48 is engaged with the transmission structure 18. The movement and engagement of the first piston 48 and the transmission structure 18 causes the distal movement of second piston 60. Due to the seal between the first piston 48 and the delivery tube 62 and the fixed nature of the delivery tube 62 relative to the movement of the first and second pistons 48, 60, the distal movement of the first piston 48 causes the first fluid to leave the first chamber 46 and enter into the first channel 74 and out of first outlet 22. The distal movement of first piston 48 causes the distal movement of the second piston 60, thereby causing the second fluid to leave the second chamber 47 and enter into the second channel 76 and out of the second outlet 24. In a preferred embodiment, the first and second fluids are dispensed out of the first and second outlets 22, 24 in a 1:1 ratio. However, in other embodiments, the ratio may be different, such as 2:1, 5:1, 10:1, and so on (see FIG. 6). FIG. 2C shows the system in a generally empty position after the first and second pistons 48, 60 have been advanced substantially distally.

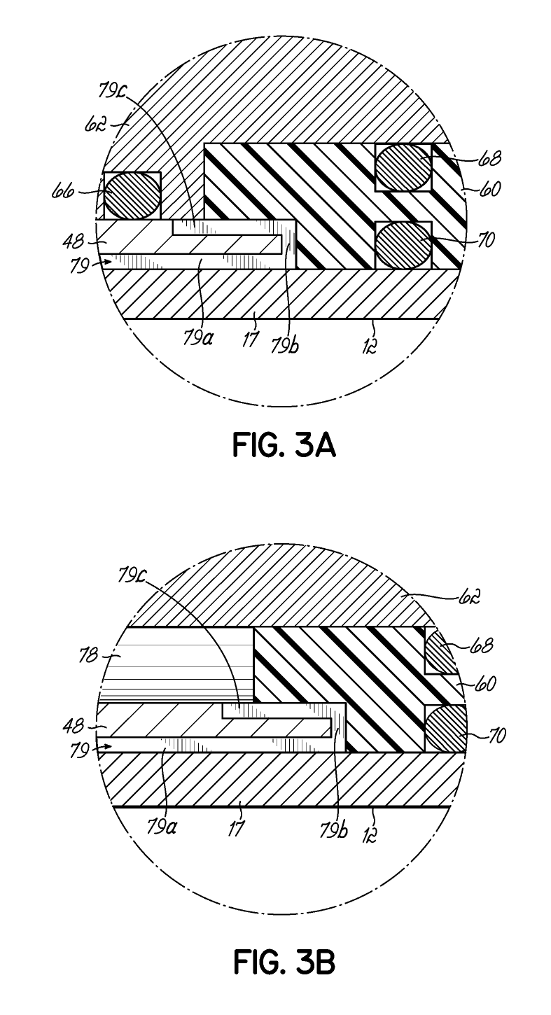

Cavity 78 increases in length as first and second pistons 48, 60 advance distally. Due to the sealed nature and configuration of the system, the cavity 78 has a natural tendency to develop a negative pressure, or become a vacuum space. To counteract the formation of a vacuum in cavity 78, a vent 79 is provided. In a preferred embodiment, vent 79 is configured as a channel along at least a portion of the length of the first piston 48, thereby preventing a vacuum from forming in cavity 78. FIG. 3A shows the vent 79 when the syringe 10 is in the filled position. Before the transmission structure 18, first piston 48 and second piston 60 advance distally, second piston 60 and delivery tube 62 may be in contact at the proximal end of the second piston 60. Vent 79 is formed as a channel on or within first piston 48 and fluidicly communicates with cavity 78 that forms between the second piston 60 and delivery tube 62 as the second piston 60 moves distally. In a preferred embodiment, the vent 79 is formed from a channel formed along at least a portion of the length of the first piston 48 and has first, second and third sections 79a, 79b, 79c. The first section 79a of the vent 79 is disposed between the first piston 48 and the wall 17 of the barrel 12. The second section 79b of the vent 79 extends radially inward and the third section 79c extends proximally away from the second section 79b and is generally parallel with the first section 79a. In an alternative embodiment, the vent 79 could be configured in any alternative manner that would counteract pressure changes in the cavity 78. As shown in FIGS. 2B, 2C and 3B, as the second piston 60 moves distally with respect to the delivery tube 62, the fluidic connection between the vent 79 and cavity 78 allows air to be drawn into cavity 78 from a space outside the system, thereby preventing negative pressure, or a vacuum, from forming in cavity 78.

Lubricious coatings may need to be provided between and among components due to the seals provided by the O-rings 66, 68, 70, 72. For example, to allow the traversal of the first and second pistons 48, 60 within the barrel 12 and past or along the delivery tube 62, lubricious coatings may be provided on the O-rings themselves or on the components with which they come into contact. For the same purpose, a lubricious coating may be provided on the transmission structure 18 and/or the barrel 12 for the traversal of the transmission structure 18 within the barrel 12.

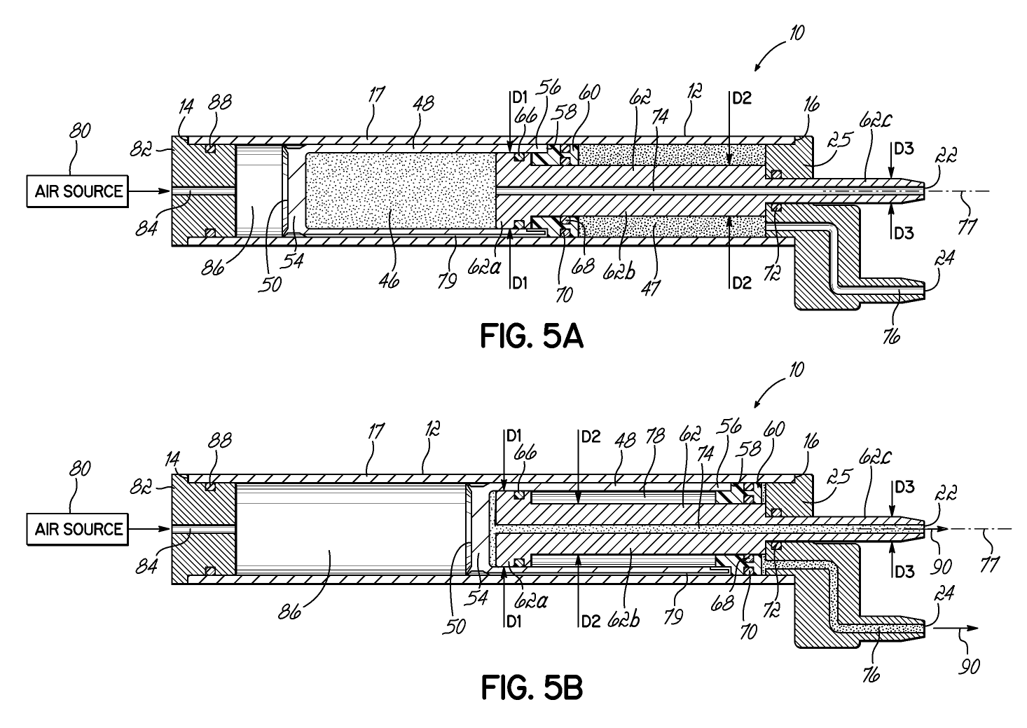

FIGS. 4, 5A and 5B show an alternative embodiment where the transmission structure 18 is a pneumatic air source 80. In this embodiment, the proximal end 14 of the barrel 12 is sealed by a pneumatic adaptor 82 having a bore 84 fluidicly communicating with air chamber 86. A fifth o-ring 88 may be provided in this embodiment to create a seal between the pneumatic adaptor 82 and the barrel 12. As seen in FIG. 5A, as air from the air source 80 is pumped into air chamber 86, the pressure within air chamber 86 increases, thereby causing the distal movement of the first piston 48 and increasing the length of the air chamber 86. The movement of the first piston 48 causes the distal movement of second piston 60. Due to the seal between the first piston 48 and the delivery tube 62 and the fixed nature of the delivery tube 62 relative to the movement of the first and second pistons 48, 60, the distal movement of the first piston 48 causes the first fluid to leave the first chamber 46 and enter into the first channel 74 and out of first outlet 22. The distal movement of first piston 48 causes the distal movement of the second piston 60, thereby causing the second fluid to leave the second chamber 47 and enter into the second channel 76 and out of the second outlet 24 in the direction of arrows 90. FIG. 5B shows the piston system in a plunged state after the first and second pistons 48, 60 have advanced distally.

As shown in FIGS. 6A and 6B, in some embodiments, the ratio between the volumes of the first and second chambers 46a, 47a may be different. In FIGS. 6A and 6B, the ratio of fluid volume in the first and second chambers 46a and 47a is 11:1. This ratio is particularly advantageous in applications using blood-based biomaterials. In other embodiments, however, the ratio of volume between the first and second chambers 46a, 47a may be different, depending on the specific application and fluids. In one embodiment, the first chamber 46a contains a clotting agent, such as thrombin. In the second chamber 47a may be blood or a blood-based material without clotting agents therein.

In the embodiment shown in FIGS. 6A and 6B, delivery tube 62′ includes first and second sections 62a′, 62b′ having first and second diameters D1 and D2, respectively. First end 54 of the first piston 48, the legs 48a, 48b of the first piston 48, and the first section 62a′ of the delivery tube 62′ define the first chamber 46a. Similar to previously disclosed embodiments, when the transmission structure 18 moves distally, the first end 54 of the first piston 48 engages with the transmission structure 18. The movement and engagement of the first piston 48 and the transmission structure 18 causes the distal movement of second piston 60. Due to the seal between the legs 48a, 48b of first piston 48 and the delivery tube 62′, and also due to the fixed nature of the delivery tube 62′ relative to the first and second pistons 48, 60, the distal movement of the first piston 48 causes the first fluid to leave the first chamber 46 and enter into the first channel 74 and out of first outlet 22. The distal movement of first piston 48 causes the distal movement of the second piston 60, thereby causing the second fluid to leave the second chamber 47 and enter into the second channel 76 and out of the second outlet 24. In a preferred embodiment, the first and second fluids are dispensed out of the first and second outlets 22, 24. In one embodiment, the fluids may be mixed after being dispensed out of the first and second outlets 22, 24. Because the blood or blood-based material tends to clot when mixed with a clotting agent such as thrombin, the mixture of the first and second fluids may be used as a tissue barrier when mixed upon exiting the first and second outlets 22, 24. However, the embodiment shown in FIGS. 6A and 6B is not limited to applications using blood or blood-based material and could be used in any application where different ratios of multiple fluids are desired.

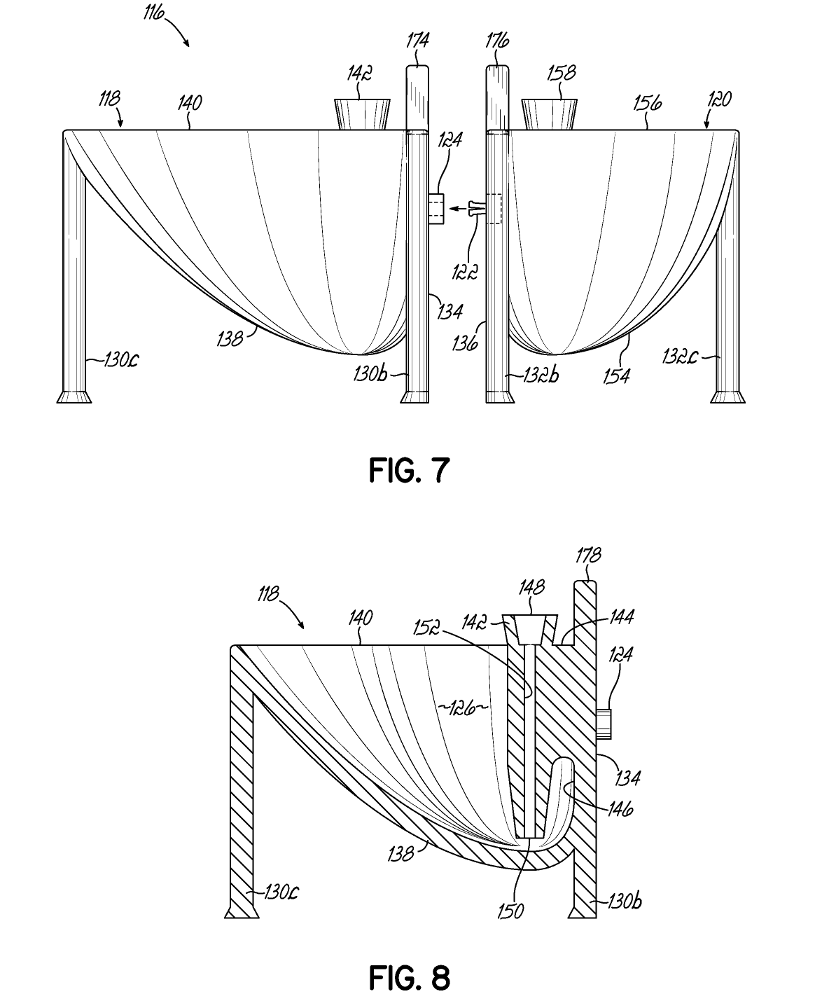

Referring to FIGS. 7-10C, part of a system 110 for holding and dispensing first and second fluids 112, 114 is shown as a container 116. The container 116 includes first and second compartments 118, 120 which are configured to be releasably attached such that the container 116 may be selectively assembled and disassembled. For example, the first compartment 118 is configured to receive and store the first fluid 112, while the second compartment 120 is configured to receive the second fluid 114. First and second fluids 112, 114 are preferably different fluids, as described above, but may alternatively be the same fluid. Compartmentalizing and allowing selective assembly and disassembly of the container 116 provides benefits such as reducing the likelihood of premature mixing of the first and second fluids 112, 114. This is undesirable because, as discussed above, the first and second fluids 112, 114 may react, or coagulate, once mixed. Moreover, such features also provide for faster filling at the clinical site, point of manufacture, or at another stage. Other benefits, such as alternating which combinations and ratios of first and second fluids 112, 114 are used with the system, as described above, are also provided.

As shown, in one embodiment, second compartment 120 includes a male feature 122 that may be received into a female feature 124 on first compartment 118. Male feature 122 is able to be snap-fitted into or with female feature 124 to releasably attach first and second compartments 118, 120 to form an essentially unitary container. It will be appreciated that one or both of the first or second compartments 118, 120 may include such features that allow the selective assembly and disassembly of container 116 and the invention is not limited to the configuration shown. Alternatively, the container 116 may be manufactured as one unitary piece. Moreover, while the first compartment 118 is shown to be larger than the second compartment 120, the relative sizes and shapes of the first and second compartments 118, 120 are not so limited. For example, the shapes of the walls surrounding and defining each cavity 126, 128 are not limited to those shown.

The first compartment 118 includes a plurality of legs 130a-c. The second compartment 120 includes a plurality of legs 132a-c. Legs 130a-c, 132a-c provide support for the compartments 118, 120. Each set of legs 130a-c, 132a-c, respectively, are spaced angularly from one another. Legs 130a-b are essentially an extension of side wall 134. Similarly, legs 132a-b are essentially an extension of side wall 136. Depending on the shape of the compartments, legs 130a-c and/or legs 132a-c may be optional. For example, the compartments 118, 120 may include a rectilinear shape such that bottom portions thereof may lie flat against a support surface such that the supporting legs 130a-c, 132a-c are unnecessary.

The first compartment 118 includes a substantially planar first side wall 134, a second, generally curvilinear wall 138, and a top edge 140 thereby defining the first fluid receiving space therebetween, also referred to herein as first cavity 126. First cavity 126 is configured for receiving and holding fluids. A first port 142 extends from a point above the top edge and into the first cavity 126, and provides communication between the first cavity 126 and an area outside of the first cavity 126 so that fluids may be directed into first cavity 126. The first port 142 is supported by a flange 144 extending into the second cavity 128 from an inner portion 146 of side wall 134. The first port 142 includes a first open end 148 residing outside of the first cavity 126, a second open end 150 residing in the first cavity 126, and a lumen 152 between the first and second ends 148, 150.

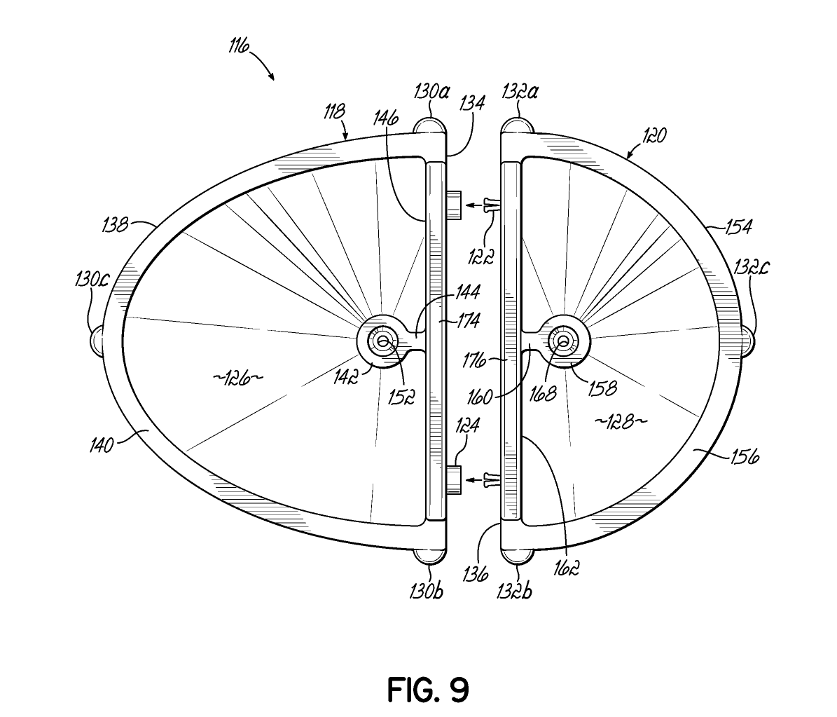

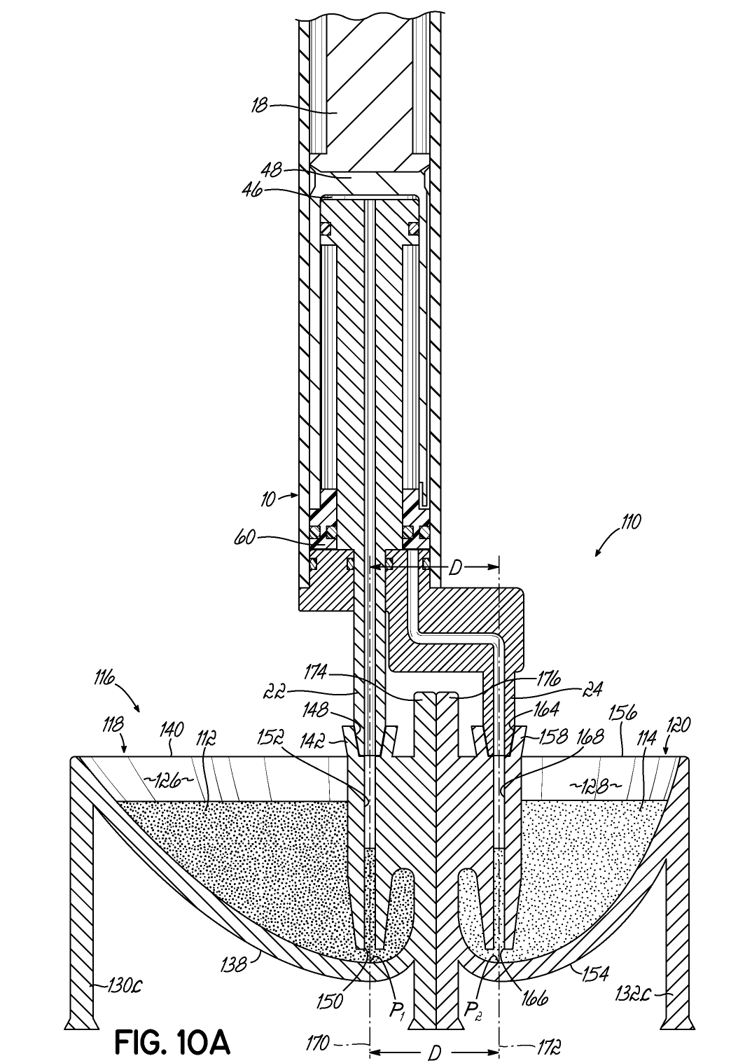

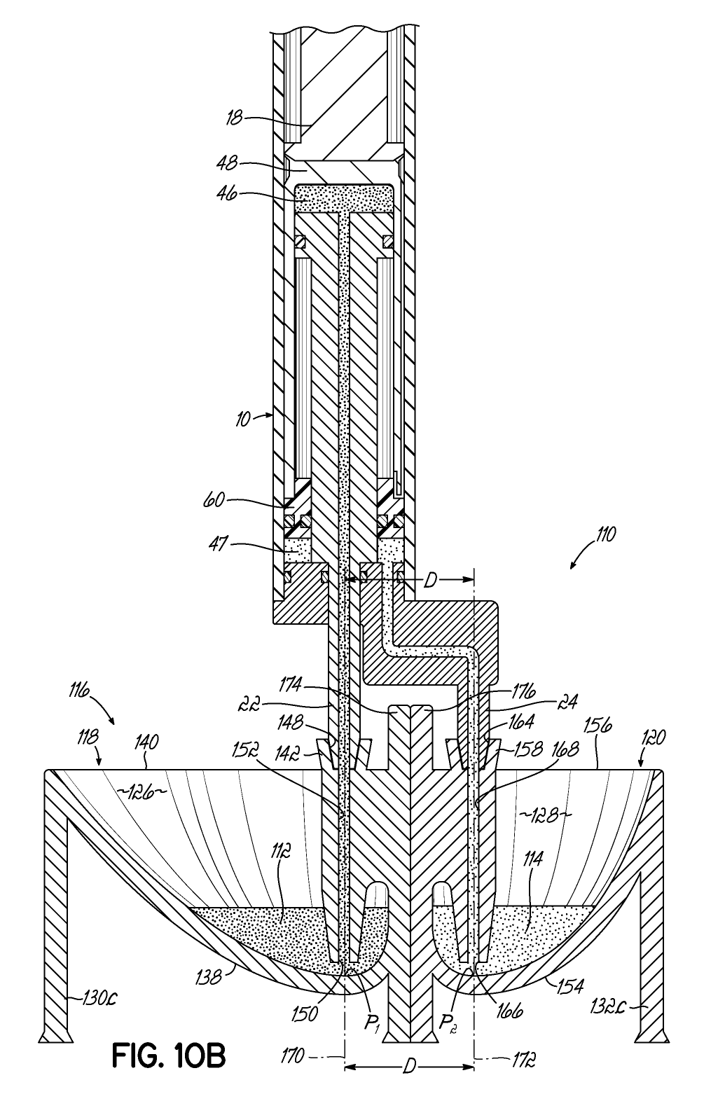

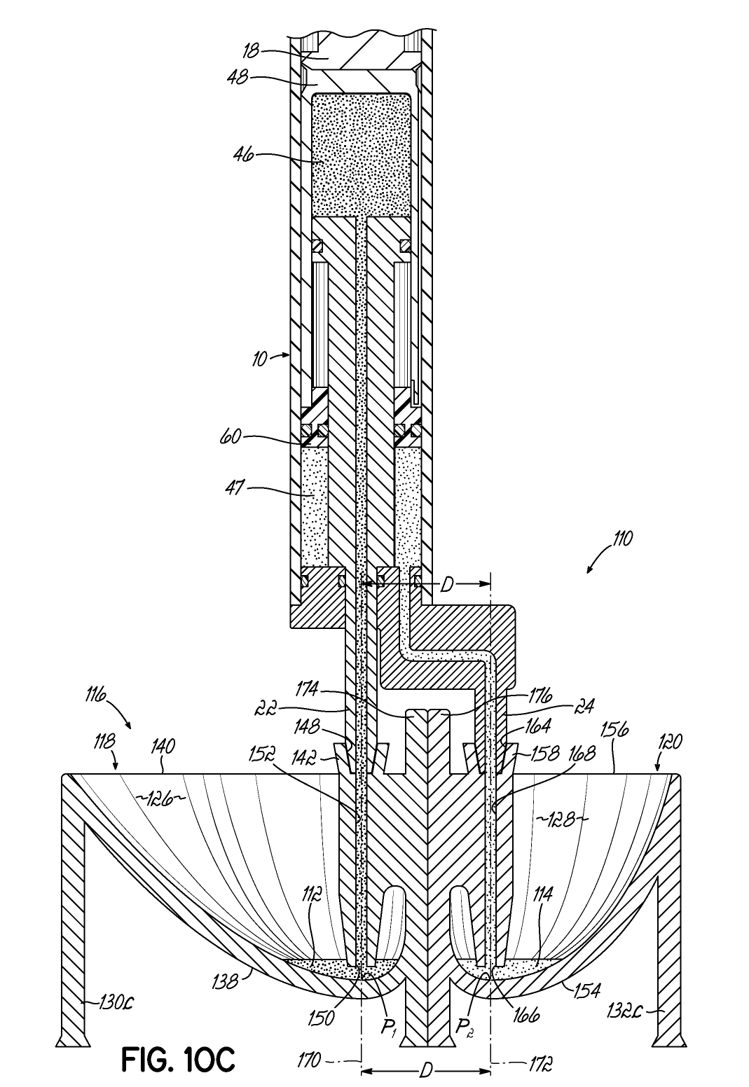

Similarly, the second compartment 120 also includes a substantially planar first side wall 136, a second, generally curvilinear wall 154, and a top edge 156 thereby defining a second fluid receiving space therebetween, also referred to herein as second cavity 128. A second port 158 extends from a point above the top edge 156 into the second cavity 128 and provides communication between the second cavity 128 and an area outside of the second cavity 128 so that fluids may be directed into second cavity 128. The second port 158 is supported by a flange 160 (FIG. 9) extending into the second cavity 128 from an inner portion 162 of the side wall 136. The second port 158 includes a first open end 164 (FIG. 10A) residing outside of the second cavity 128, a second open end 166 residing in the second cavity 128, and a lumen 168 between the first and second open ends 164, 166. While the first cavity 126 is shown to be larger than the second cavity 128, the relative sizes of the first and second cavities 126, 128 are not so limited and may be different depending on the desired ratios of the first and second fluids 112, 114 as described herein. While the first and second compartments 118, 120 are shown without lids or top walls, alternative embodiments of the compartments may include lids or top walls such that the fluids may be advantageously pre-filled and stored within the compartments 118, 120 as described herein. The first open ends 148, 164 of the first and second ports 142, 158 may each include female luer connections (not shown), while the first and second outlets 22, 24 each may then include corresponding male luer connections (not shown) for fluid connection purposes.

In the embodiment shown, first and second cavities 126, 128 are advantageously shaped such that a cross-sectional shape of at least a portion of each is defined by an at least partially parabolic curve. Such a shape is advantageous in that, where an amount of fluid in either cavity 126, 128 is relatively low, the remaining amount of fluid will naturally collect in the deepest regions, i.e., point P₁ and P₂ of each cavity 126, 128, respectively (FIG. 10A). Moreover, a sharper curvature at such a region can provide for deeper pooling of the fluid at the lowest region. For at least this reason, each of the first and second axes 170, 172 extending through the center of each port essentially intersects the deepest point P₁, P₂ of each cavity 126, 128 such that a maximum amount of fluid may be drawn into each lumen 152, 168, out of each respective cavity 126, 128 through second open ends 150, 166 of ports 142, 158.

A splash guard 174, 176 is provided on each of the first and second compartments 118, 120, respectively. Each splash guard 174, 176 is essentially an extension of a respective side wall 134, 136, but in alternative embodiments may be a separate component that can be selectively attachable and detachable from its respective compartment. As best shown in FIG. 9, each splash guard 174, 176 extends along a substantial portion of the length of each respective side wall 134, 136. However, the splash guards 174, 176 are not limited to such dimensions and may be shorter or longer, and include a smaller or larger width and a smaller or larger height. For example, instead of traversing almost the entire length of the side wall 134, 136, the splash guards 174, 176 may simply traverse a portion at or near each of their respective ports 142, 158.

The splash guards 174, 176 prevent unwanted mixing of the first and second fluids 112, 114 as the fluids are directed into or drawn from the first and second compartments 118, 120, respectively. The splash guards 174, 176 may guard against events such as one or both of the fluids 112, 114 splashing, being spilled or flowing across to the other compartment 118, 120 or any similar occurrence that may lead to unwanted mixing of the first and second fluids 112, 114. While each of the first and second compartments 118, 120 is shown to have a splash guard 174, 176, alternatively, only one, or neither, of the compartments may include a splash guard.

Referring specifically to FIGS. 10A-C, the system 110 is shown with the syringe 10 and the container 116 assembled. While the container 116 described herein is able to be used with many different syringes having multiple fluid chambers, the syringe 10 described with respect to the system 110 may be essentially the same as that described with respect to FIGS. 1-3B and, for that reason, the same reference numerals are used to describe the like or identical structures.

Regarding the container 116, first and second compartments 118, 120 are removably attached to one another. As shown, first and second outlets 22, 24 of syringe 10 extend along first and second axes 170, 172, respectively, which are spaced apart by first distance D. In order to facilitate easy connection between the ports 142, 158 and the outlets 22, 24 without additional parts, such as tubing or other structure used for fluidic connection, the first and second ports 142, 158 are positioned substantially coaxially with the first and second outlets 22, 24 when the first and second compartments 118, 120 are attached to each other. In other words, the first and second ports 142, 158 are also spaced apart by the first distance D. As shown, the reference points for measuring the first distance D are the centers of the first and second ports 142, 158, respectively, but it will be appreciated that the reference point(s) for measuring the first distance D with respect to the first and second outlets 22, 24 as well as the first and second ports 142, 158 may be different. The system 110 may be so designed that, regardless of the shape and size of each of the compartments 118, 120, the first and second ports 142, 158 may be positioned to be spaced apart by the first distance D when the first and second compartments 118, 120 are attached such that the syringe 10 and container 116 may be fluidicly connected as described herein. Of course, the first distance D is not so limited to the distance shown.

First cavity 126 of first compartment 118 is filled with first fluid 112 and second cavity 128 of second compartment 120 is filled with the second fluid 114. The compartments 118, 120 may arrive at the clinical site or other site of use pre-filled such that they are filled with their respective fluids at a site of manufacture or prior to the entering the clinical or other site. Alternatively, a clinician may fill one or both of the cavities 126, 128 with their respective fluids in the clinical environment. Moreover, the compartments 118, 120 may be coupled when they arrive at the clinical site or alternatively, may be coupled after the clinician chooses from a selection of first and second compartments 118, 120 having a desired ratio of volumes of first and second fluids 112, 114. The clinician's choice may depend on the desired ratios of first fluid 112 to second fluid 114, which also may depend on, among other things, the respective sizes of respective first and second fluid chambers 46, 47 of the syringe 10.

First outlet 22 of syringe or cartridge 10 is in fluidicly connected to first port 142 and second outlet 24 is fluidicly connected to second port 158. Once each port 142, 158 is in fluid communication with its respective outlet 22, 24 (FIG. 10A), fluid may be drawn into the first and second fluid chambers 46, 47. Essentially, drawing fluid into the first and second fluid chambers 46, 47 occurs in a manner opposite to directing fluid out of the first and second fluid chambers 46, 47 as described herein. FIG. 10A shows the syringe 10 in a generally empty state, and first and second pistons 48, 60 are positioned in a substantially distal position. In order to move the first and second pistons 48, 60 in the proximal direction, transmission structure 18 such as a plunger is drawn in the proximal direction. Such movement of the pistons 48, 60 draws the fluids 112, 114 into each respective outlet 22, 24, and further into the first and second fluid chambers 46, 47, respectively, until the first and second chambers 46, 47 are filled to a desired amount. Notably, as the syringe 10 described herein is configured to dispense fluids 112, 114 at a specified ratio by depression of the plunger or transmission structure 18, the syringe 10 is also configured to draw fluids therein at a specified ratio. Once a certain amount of the first and second fluids 112, 114 is drawn into the first and second fluid chambers 46, 47, the ports 142, 158 and outlets 22, 24 may be disconnected and the syringe 10 may be used as described herein. For example, the system 110 may further include a cannula assembly 26 (FIG. 1) coupled with and fluidicly communicating with the first and second outlets 22, 24, such that the first and second fluids 112, 114 may be dispensed from a tip 39 of the cannula assembly 26.

It will be appreciated that, generally, when drawing fluids into a syringe, drawing an amount of air into the one or more fluid chambers may be inevitable. Therefore, the drawings shown may be exaggerated in that no air is shown as being drawn into the first and second fluid chambers 46, 47 as the transmission structure 18 is moved proximally. However, it will be understood that the air introduced into the fluid chambers 46, 47 may be purged as understood in the art by, for example, inverting the syringe 10 and advancing the transmission structure 18 so as to purge the air from the syringe 10. Because the outlets 22, 24 are of different lengths, the fluid resistance in the second outlet 24 is greater than the first outlet 22. Without equalizing the fluid resistance of each outlet 22, 24, one of the chambers 46, 47 may be purged of air before the other and fluid may be dispensed in an unwanted manner. In this regard, the diameter of one or both of the outlets 22, 24 may be altered such that the fluid resistance of each outlet is identical. For example, a cross sectional dimension (i.e., diameter) of the longer second outlet 24 may be increased, or a cross-sectional dimension of the first outlet 22 may be decreased, so that the fluid resistance thereof equals the resistance of the other.

While the present invention has been illustrated by the description of one or more embodiments thereof, and while the embodiments have been described in considerable detail, they are not intended to restrict or in any way limit the scope of the appended claims to such detail. Additional advantages and modifications will readily appear to those skilled in the art. The invention in its broader aspects is therefore not limited to the specific details, representative apparatus and method and illustrative examples shown and described. Accordingly, departures may be from such details without departing from the scope or spirit of the general inventive concept. What is claimed is:

1. A system for dispensing first and second fluids, comprising:
a syringe including:
a first fluid chamber;
a second fluid chamber;
a first outlet communicating with the first fluid chamber;
a second outlet communicating with the second fluid chamber;
a transmission structure operative to draw the first and second fluids into the first and second outlets and further into the first and second fluid chambers, respectively, and to dispense the first and second fluids from the first and second outlets, respectively; and
a container having first and second compartments separated by at least one wall and configured to respectively store the first and second fluids, the first compartment having a first port positioned to be fluidicly connected with the first outlet and the second compartment having a second port positioned to be fluidicly connected with the second outlet.

2. The system of claim 1, wherein the first outlet extends along a first axis and the second outlet extends along a second axis, the second axis spaced from the first axis by a first distance, and the first and second ports are spaced apart by the first distance.

3. The system of claim 1, further comprising:
a cannula assembly coupled with and fluidicly communicating with the first and second outlets, the cannula assembly having a tip at a distal portion thereof, the tip adapted to dispense the first and second fluids from the cannula assembly.

4. The system of claim 1, wherein the first and second compartments are configured to be releasably attached such that the container may be selectively assembled and disassembled.

5. The system of claim 4, wherein the first and second containers are coupled together with a snap fit.

6. The system of claim 1, wherein the volume of the first compartment is greater than or equal to the volume of the first fluid chamber.

7. The system of claim 1, wherein the volume of the second compartment is greater than or equal to the volume of the second fluid chamber.

8. The system of claim 1, further comprising at least one splash guard between the first and second ports to prevent unintended mixing of the first and second fluids.

9. The system of claim 1, wherein at least one of the first or second ports includes a first open end communicating with an area outside of its respective compartment, a second open end communicating with its respective compartment, and a lumen therebetween.

10. The system of claim 9, wherein the second open end of at least one of the first or second ports is substantially aligned with or adjacent to a deepest point of its respective first or second compartment.

11. The system of claim 9, wherein the lumen extends along an axis that intersects a deepest point of its respective first or second compartment.

12. A method of dispensing first and second fluids using a system including a syringe and a container, the syringe including a first fluid chamber and a second fluid chamber, and the container including first and second compartments respectively holding the first and second fluids, comprising:
coupling the first chamber in fluid communication with the first compartment;
coupling the second chamber in fluid communication with the second compartment;
simultaneously drawing the first fluid from the first compartment into the first chamber and the second fluid from the second compartment into the second chamber;
decoupling the first and second chambers from the first and second compartments; and
simultaneously dispensing the first and second fluids from the first and second chambers.

13. The method of claim 12, further comprising:
releasably attaching the first and second compartments together prior to simultaneously drawing the first and second fluids.

14. The method of claim 13, wherein releasably attaching the first and second compartments includes snap fitting the first and second compartments together.

15. The method of claim 12, wherein simultaneously drawing the first and second fluids further comprises:
positioning a first open end of a first lumen at a deepest point of the first compartment;
positioning a second open end of a second lumen at a deepest point of the second compartment; and
simultaneously drawing the first and second fluids respectively through the first and second lumens from the deepest points of the first and second compartments.

16. The method of claim 12, wherein coupling the first and second chambers in fluid communication with the first and second compartments further comprises:
coupling a first outlet of the syringe to a first port of the container extending along a first axis; and
coupling a second outlet of the syringe to a second port of the container extending along a second axis.
